(19) United States
(12) Patent Application Publication
(10) Pub. No.: US 2009/0204087 A1
(43) Pub. Date: Aug. 13, 2009

(54) Superabsorbent Polymers Having Superior Gel Integrity, Absorption Capacity, and Permeability

(75) Inventors: Norbert Herfert, Altenstadt (DE); Martin Wendker, Worms (DE); Rich Goodwin, Suffolk, VA (US); Ma-Ikay Kikama Miatudila, Monroe, NC (US); Michael A. Mitchell, Waxhaw, NC (US); William G-J Chiang, Yorktown, VA (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: BASF SE, Ludwigshafen (DE)

(21) Appl. No.: 12/438,839

(22) PCT Filed: Aug. 7, 2007
(86) PCT No.: PCT/EP2007/058177

§ 371 Date: Feb. 25, 2009

Related U.S. Application Data
(60) Provisional application No. 60/841,411, filed on Aug. 31, 2006.

(51) Int. Cl. A61F 13/531; C08F 20/06; B01J 20/26; A61F 13/534; A61F 13/537

(52) U.S. Cl. 604/368; 526/317.1; 502/402; 604/378

(57) ABSTRACT

Superabsorbent polymer particles having superior gel integrity, absorption capacity, and permeability are disclosed. A method of producing the superabsorbent polymer particles by applying a polyamine coating to the particles also is disclosed.

### FIELD OF THE INVENTION

The present invention relates to superabsorbent polymer particles having improved gel integrity, absorption capacity, and permeability properties. The present invention also relates to methods of manufacturing the superabsorbent polymer particles from surface-crosslinked superabsorbent polymer particles, a polyamine, water, an optional cosolvent having hydroxy groups, and an optional crosslinking agent. The polyamine-coated particles exhibit an excellent gel bed permeability and gel integrity essentially without adversely affecting absorption properties. In some embodiments, the superabsorbent polymer particles also have a reduced tendency to agglomerate. The present invention also relates to the use of the polyamine-coated superabsorbent polymer particles in articles, such as diapers, catamenial devices, and wound dressings.

### BACKGROUND OF THE INVENTION

Water-absorbing resins are widely used in sanitary goods, hygienic goods, wiping cloths, water-retaining agents, dehydrating agents, sludge coagulants, disposable towels and bath mats, disposable door mats, thickening agents, disposable litter mats for pets, condensation-preventing agents, and release control agents for various chemicals. Water-absorbing resins are available in a variety of chemical forms, including substituted and unsubstituted natural and synthetic polymers, such as hydrolysis products of starch acrylonitrile graft polymers, carboxymethylcellulose, crosslinked polyacrylates, sulfonated polystyrenes, hydrolyzed polyacrylamides, polyvinyl alcohols, polyethylene oxides, polyvinylpyrrolidones, and polyacrylonitriles. The most commonly used SAP for absorbing electrolyte-containing aqueous fluids, such as urine, is neutralized polyacrylic acid, e.g., containing about 50% and up to 100%, neutralized carboxyl groups.

Such water-absorbing resins are termed “superabsorbent polymers or SAPs, and typically are lightly crosslinked hydrophilic polymers. SAPs are generally discussed in Goldman et al. U.S. Pat. Nos. 5,669,894 and 5,599,335, each incorporated herein by reference. SAPs can differ in their chemical identity, but all SAPs are capable of absorbing and retaining amounts of aqueous fluids equivalent to many times their own weight, even under moderate pressure. For example, SAPs can absorb one hundred times their own weight, or more, of distilled water. The ability to absorb aqueous fluids under a confining pressure is an important requirement for an SAP used in a hygienic article, such as a diaper.

As used herein, the terms “base polymer particles “surface-crosslinked SAP particles and “SAP particles” refer to superabsorbent polymer particles in the dry state, i.e., particles containing from no water up to an amount of water less than the weight of the particles. “Base polymer particles” are SAP particles prior to a surface-crosslinking process. “Surface-crosslinked SAP particles” are base polymer particles that have been subjected to a surface-crosslinking process, as described more fully hereafter. The term “particles” refers to granules, fibers, flakes, spheres, powders, platelets, and other shapes and forms known to persons skilled in the art of superabsorbent polymers. The terms “SAP gel” and “SAP hydrogel” refer to a super-absorbent polymer in the hydrated state, i.e., particles that have absorbed at least their weight in water, and typically several times their weight in water. The term “coated SAP particles” and “coated surface-crosslinked polymer particles” refer to particles of the present invention, i.e., surface-crosslinked SAP particles having a polyamine coating comprising a polyamine and an optional crosslinking agent.

The terms “surface treated” and “surface crosslinked” refer to an SAP, i.e., base polymer, particle having its molecular chains present in the vicinity of the particle surface crosslinked by a compound applied to the surface of the particle. The term “surface crosslinking” means that the level of functional crosslinks in the vicinity of the surface of the base polymer particle generally is higher than the level of functional crosslinks in the interior of the base polymer particle. As used herein, “surface” describes the outer-facing boundaries of the particle. For porous SAP particles, exposed internal surface also are included in the definition of surface.

The term “polyamine coating” refers to a coating on the surface of an SAP particle, wherein the coating comprises (a) a polymer containing at least two, and typically a plurality, of primary, and/or secondary, and/or tertiary, and/or quaternary nitrogen atoms, (b) water, (c) an optional cosolvent, and (d) an optional crosslinking agent. At least a portion of the water and optional cosolvent typically evaporate from the coating during the step of applying the coating to the SAP particles. The cosolvent is capable of transforming the polyamine-coated SAP surface from hydrophilic to hydrophobic.

SAP particles can differ in ease and cost of manufacture, chemical identity, physical properties, rate of water absorption, and degree of water absorption and retention, thus making the ideal water-absorbent resin a difficult compound to design. For example, the hydrolysis products of starch-acrylonitrile graft polymers have a comparatively high ability to absorb water, but require a cumbersome process for production and have the disadvantages of low heat resistance and decay or decomposition due to the presence of starch. Conversely, other water-absorbent polymers are easily and cheaply manufactured and are not subject to decomposition, but do not absorb liquids as well as the starch-acrylonitrile graft polymers.

Therefore, extensive research and development has been directed to providing a method of increasing the fluid absorption properties of stable, easy-to-manufacture SAP particles to match the superior fluid absorption properties of difficult-to-manufacture particles. Likewise, it would be advantageous to further increase the fluid absorption properties of already-superior SAP particles.

This is a difficult goal to achieve because improving one desirable property of an SAP particle often adversely affects another desirable property of the SAP particle. For example, absorptivity and gel permeability are conflicting properties. Therefore, a balanced relation between absorptivity and gel permeability is desired in order to provide sufficient liquid absorption, liquid transport, and dryness of the diaper and the skin when using SAP particles in a diaper.

In this regard, not only is the ability of the SAP particles to retain a liquid under subsequent pressure an important property, but absorption of a liquid against a simultaneously acting pressure, i.e., during liquid absorption, also is important. This is the case in practice when a child or adult sits or lies on a sanitary article, or when shear forces are acting on the sanitary article, e.g., leg movements. This absorption property is referred to as absorption under load.

The current trend in the hygiene sector, e.g., in diaper design, is toward ever thinner core constructions having a reduced cellulose fiber content and an increased SAP content. This is an especially important trend in baby diapers and adult incontinence products. As diaper cores become thinner, the SAP particles must possess properties that historically have been supplied by fluff pulp. For example, fluid intake by a diaper core is enhanced by a higher ratio of fluff to SAP. Also, the integrity of the core is better when a higher ratio of fibrous fluff to SAP is utilized.

This trend has substantially changed the performance profile required of SAPs. Whereas SAP development initially was focused on very high absorption and swellability, it subsequently was determined that an ability of SAP particles to transmit and distribute a fluid both into the particle and through a bed of SAP particles also is of major importance. Conventional SAPs undergo great surface swelling when wetted with a fluid, such that transport of the fluid into the particle interior is substantially compromised or completely prevented. Historically, a substantial amount of cellulose fibers has been included in a diaper core to quickly absorb the fluid for eventual distribution to the SAP particles, and to physically separate SAP particles in order to prevent fluid transport blockage.

An increased amount of SAP particles per unit area in a hygiene article must not cause the swollen polymer particles to form a barrier layer to absorption of a subsequent fluid insult. Therefore, an SAP having good permeability properties ensures optimal utilization of the entire hygiene article. This prevents the phenomenon of gel blocking, which in the extreme case causes the hygiene article to leak. Fluid transmission and distribution, therefore, is of maximum importance with respect to the initial absorption of body fluids.

However, because the absorption properties and permeability properties of SAP particles are conflicting, it is difficult to improve one of these properties without adversely affecting the other property. Investigators have researched various methods of improving the amount of fluid absorbed and retained by SAP particles, especially under load, and the rate at which the fluid is absorbed. One preferred method of improving the absorption and retention properties of SAP particles is to surface treat the SAP particles.

The surface treatment of SAP particles with crosslinking agents having two or more functional groups capable of reacting with pendant carboxylate groups on the polymer comprising the SAP particle is disclosed in numerous patents. Surface treatment improves absorbency and gel rigidity to increase fluid flowability and prevent SAP particle agglomeration, and improves gel strength.

Surface-crosslinked SAP particles, in general, exhibit higher liquid absorption and retention values than SAP particles having a comparable level of internal crosslinks, but lacking surface crosslinks. Internal crosslinks arise from polymerization of the monomers comprising the SAP particles, and are present in the polymer backbone. It has been theorized that surface crosslinking increases the resistance of SAP particles to deformation, thus reducing the degree of contact between surfaces of neighboring SAP particles when the resulting hydrogel is deformed under an external pressure. The degree to which absorption and retention values are enhanced by surface crosslinking is related to the relative amount and distribution of internal and surface crosslinks, and to the particular surface-crosslinking agent and method of surface crosslinking.

The present invention is directed to surface-crosslinked SAP particles that are coated with a polyamine, water, an optional cosolvent, and an optional crosslinking agent. The coated SAP particles demonstrate an improved gel bed permeability (GBP) and gel integrity (GI) without a substantial adverse affect on the fluid absorbency properties (e.g., centrifuge retention capacity (CRC)) of the SAP particles.

### SUMMARY OF THE INVENTION

The present invention is directed to surface-crosslinked SAP particles having a superior gel integrity, absorption capacity, and permeability. More particularly, the present invention is directed to surface-crosslinked SAP particles having a coating comprising a polyamine, water, an optional cosolvent, and an optional crosslinking agent hereafter referred to as a “polyamine coating.” At least a portion of the water, and often a portion of the optional cosolvent, typically evaporate from the coating under the conditions of curing the polyamine coating on the surface-crosslinked SAP particles. The present invention also is directed to methods of preparing the polyamine-coated SAP particles. The polyamine surface coating can be hydrophilic or hydrophobic.

One aspect of the present invention is to provide surface-crosslinked SAP particles having an excellent gel bed permeability, a high absorbance under load, a good gel integrity, and a high centrifuge retention capacity, and that also demonstrate an improved ability to absorb and retain electrolyte-containing fluids, such as saline, blood, urine, and menses.

Another aspect of the present invention is to provide polyamine-coated, surface-crosslinked SAP particles having the above-listed properties and a reduced tendency to agglomerate. The polyamine coating is applied after surface crosslinking of the SAP particles is complete.

Still another aspect of the present invention is to prepare coated SAP particles of the present invention by applying an aqueous polyamine solution, optional cosolvent, and optional crosslinking agent, individually or in admixture, to the surfaces of the surface-crosslinked SAP particles at a temperature of about 25° C. to about 100° C., and mixing for about 5 to about 60 minutes.

Yet another aspect of the present invention is to provide polyamine-coated, surface-cross-linked SAP particles having a hydrophobic surface. Such SAP particles have a reduced tendency to agglomerate. The polyamine coated surface is rendered hydrophobic by including a cosolvent in the polyamine coating process. The particles have a reduced tendency to agglomerate compared to identical SAP particles coated with a polyamine in the absence of a cosolvent.

Another aspect of the present invention is to provide polyamine-coated, surface-crosslinked SAP particles having a centrifuge retention capacity (CRC) of at least about 25 g/g (gram/gram), a gel integrity (GI) of at least 2, a free swell gel bed permeability of at least 200 Darcies, and preferably a gel bed permeability (GBP) (0.3 psi) of at least 3 Darcies, while retaining an excellent absorbance under load (AUL). Surprisingly, these absorption, permeability, and gel integrity properties are essentially independent of the fluid wicking index of the polyamine-coated SAP particles.

Still another aspect of the present invention is to provide absorbent hygiene articles, such as diapers, having a core comprising polyamine-coated SAP particles of the present invention. The diaper cores typically contain greater than 50%, by weight, of the present polyamine-coated SAP particles.

Another aspect of the present invention is to provide absorbent hygiene articles having a core containing a relatively high concentration of polyamine-coated SAP particles, which provide improved gel permeability and gel integrity, essentially without a decrease in absorbent properties, and preferably have a reduced tendency to agglomerate.

These and other aspects and advantages of the present invention will become apparent from the following detailed description of the preferred embodiments.

### DETAILED DESCRIPTION OF THE PREFERRED EMBODIMENTS

The present invention is directed to surface-crosslinked SAP particles coated with a polyamine, water, an optional cosolvent, and an optional crosslinking agent. SAPs for use in personal care products to absorb body fluids are well known. SAP particles typically are polymers of unsaturated carboxylic acids or derivatives thereof. These polymers are rendered water insoluble, but water swellable, by crosslinking the polymer with a di- or polyfunctional internal crosslinking agent. These internally crosslinked polymers are at least partially neutralized and contain pendant anionic carboxyl groups on the polymer backbone that enable the polymer to absorb aqueous fluids, such as body fluids. Typically, the SAP particles are subjected to a post-treatment to crosslink the pendant anionic carboxy groups on the surface of the particle.

SAPs are manufactured by known polymerization techniques, preferably by polymerization in aqueous solution by gel polymerization. The products of this polymerization process are aqueous polymer gels, i.e., SAP hydrogels, that are reduced in size to small particles by mechanical forces, then dried using drying procedures and apparatus known in the art. The drying process is followed by pulverization of the resulting SAP particles to the desired particle size.

To improve the fluid absorption profile, SAP particles are optimized with respect to one or more of absorption capacity, absorption rate, acquisition time, gel strength, and/or permeability. Optimization allows a reduction in the amount of cellulosic fiber in a hygienic article, which results in a thinner article. However, it is difficult to impossible to maximize all of these absorption profile properties simultaneously.

One method of optimizing the fluid absorption profile of SAP particles is to provide SAP particles of a predetermined particle size distribution. In particular, particles too small in size swell after absorbing a fluid and can block the absorption of further fluid. Particles too large in size have a reduced surface area which decreases the rate of absorption.

Therefore, the particle size distribution of the SAP particles is such that fluid permeability, absorption, and retention by the SAP particles is maximized. Any subsequent process that agglomerates the SAP particles to provide oversized particles should be avoided. In particular, agglomeration of SAP particles increases apparent particle size, which reduces the surface area of the SAP particles, and in turn adversely affects absorption of an aqueous fluid by the SAP particles.

The present invention is directed to overcoming problems encountered in improving the absorption profile of surface-crosslinked SAP particles because improving one property often is detrimental to a second property. The present polyamine-coated SAP particles maintain the conflicting properties of a high centrifuge retention capacity (CRC), an excellent gel bed permeability (GBP), and a good gel integrity (GI). These problems are overcome because of the polyamine coating, and in some embodiments because of the reduced tendency of the present polyamine-coated SAP particles to agglomerate.

In order to use an increased amount of SAP particles, and a decreased amount of cellulose, in personal care products, it is important to maintain a high SAP liquid permeability. In particular, the permeability of an SAP particle hydrogel layer formed by swelling in the presence of a body fluid is very important to overcome the problem of leakage from the product. A lack of permeability directly impacts the ability of SAP particle hydrogel layers to acquire and distribute body fluids.

Polyamines are known to adhere to cellulose (i.e., fluff), and polyamine-coated SAPs have some improved permeability, as measured in the bulk, for a lower capacity SAP. Coating of SAP particles with uncrosslinked polyamines improves adhesion to cellulose fibers because of the high flexibility of polyamine molecules. Preferably, covalent bonding of the polyamine to the SAP particles is avoided because the degree of SAP particle crosslinking is increased and the absorptive capacity of the particles is reduced. Moreover, covalent bonding of polyamine to the SAP particle surface typically occurs at a temperature greater than 150° C., which adversely affects the color of the SAP particles, and, ultimately, consumer acceptance of the hygiene article.

The addition of a cationic compound, e.g., a polyamine, to improve permeability of SAP particles has been disclosed. WO 03/043670 discloses a polyamine coating on an SAP particle wherein the polyamine molecules are covalently crosslinked to one another. WO 95/22356 and U.S. Pat. No. 5,849,405 disclose an absorbent material comprising a mixture of an SAP and an absorbent property modification polymer (e.g., a cationic polymer) that is reactive with at least one component included in urine (e.g., phosphate ion, sulfate ion, or carbonate ion). WO 97/12575 also discloses the addition of a polycationic compound without further crosslinking.

Other patents disclosing incorporation of polyamine-coated superabsorbents in fibrous matrices, e.g., U.S. Pat. No. 5,641,561, U.S. Pat. No. 5,382,610, EP 0 493 011, and WO 97/39780, relate to an absorbent material having improved structural stability in the dry and wet states. The material comprises hydrogel-forming SAP particles, a polycationic polymer bonded to the absorbent particles at the surface thereof, and glue microfibers that act as an adhesive between SAP particles and the carrier layer. The carrier layer can be a woven or nonwoven material, and the polycationic polymer can be a polyamine, a polyimine, or a mixture thereof. U.S. Pat. No. 5,324,561 discloses an SAP which is directly crosslinked to amine-epichlorohydrin adducts (e.g., KYMENE® products).

In accordance with the present invention, surface-crosslinked SAP particles coated with a polyamine solution and an optional cosolvent are disclosed. The present SAP particles comprise a base polymer. The base polymer can be a homopolymer or a copolymer. The identity of the base polymer is not limited as long as the polymer is an anionic polymer, i.e., contains pendant acid moieties, and is capable of swelling and absorbing at least ten times its weight in water, when in a neutralized form. Preferred base polymers are crosslinked polymers having acid groups that are at least partially in the form of a salt, generally an alkali metal or ammonium salt.

The base polymer has at least about 25% of the pendant acid moieties, e.g., carboxylic acid moieties, present in a neutralized form. Preferably, the base polymer has about 50% to about 100%, and more preferably about 65% to about 80%, of the pendant acid moieties present in a neutralized form. In accordance with the present invention, the base polymer has a degree of neutralization (DN) of about 25 to about 100.

The base polymer of the SAP particles is a lightly crosslinked polymer capable of absorbing several times its own weight in water and/or saline. SAP particles can be made by any conventional process for preparing superabsorbent polymers and are well known to those skilled in the art. One process for preparing SAP particles is a solution polymerization method described in U.S. Pat. Nos. 4,076,663; 4,286,082; 4,654,039; and 5,145,906, each incorporated herein by reference. Another process is an inverse suspension polymerization method described in U.S. Pat. Nos. 4,340,706; 4,497,930; 4,666,975; 4,507,438; and 4,683,274, each incorporated herein by reference.

SAP particles useful in the present invention are prepared from one or more monoethylenically unsaturated compound having at least one acid moiety, such as carboxyl, carboxylic acid anhydride, carboxylic acid salt, sulfuric acid, sulfuric acid salt, sulfonic acid, sulfonic acid salt, phosphoric acid, phosphoric acid salt, phosphonic acid, or phosphonic acid salt. SAP particles useful in the present invention preferably are prepared from one or more monoethylenically unsaturated, water-soluble carboxyl or carboxylic acid anhydride containing monomer, and the alkali metal and ammonium salts thereof, wherein these monomers preferably comprise 50 to 99.9 mole percent of the base polymer.

The base polymer of the SAP particles preferably is a lightly crosslinked acrylic resin, such as lightly crosslinked polyacrylic acid. The lightly crosslinked base polymer typically is prepared by polymerizing an acidic monomer containing an acyl moiety, e.g., acrylic acid, or a moiety capable of providing an acid group, i.e., acrylonitrile, in the presence of an internal crosslinking agent, i.e., a polyfunctional organic compound. The base polymer can contain other copolymerizable units, i.e., other monoethylenically unsaturated comonomers, well known in the art, as long as the base polymer is substantially, i.e., at least 10%, and preferably at least 25%, acidic monomer units, e.g., (meth)acrylic acid. To achieve the full advantage of the present invention, the base polymer contains at least 50%, and more preferably, at least 75%, and up to 100%, acidic monomer units. The other copolymerizable units can, for example, help improve the hydrophilicity of the polymer.

Ethylenically unsaturated carboxylic acid and carboxylic acid anhydride monomers useful in the base polymer include acrylic acid, methacrylic acid, ethacrylic acid, α-chloroacrylic acid, α-cyanoacrylic acid, β-methylacrylic acid (crotonic acid), α-phenylacrylic acid, β-acryloxypropionic acid, sorbic acid, α-chlorosorbic acid, angelic acid, cinnamic acid, p-chlorocinnamic acid, β-stearylacrylic acid, itaconic acid, citraconic acid, mesaconic acid, glutaconic acid, aconitic acid, maleic acid, fumaric acid, tricarboxyethylene, and maleic anhydride.

Ethylenically unsaturated sulfonic and phosphonic acid monomers include aliphatic or aromatic vinyl sulfonic acids, such as vinylsulfonic acid, allylsulfonic acid, vinyl toluene sulfonic acid, styrene sulfonic acid, acrylic and methacrylic sulfonic acids, such as sulfoethyl acrylate, sulfoethyl methacrylate, sulfopropyl acrylate, sulfopropyl methacrylate, 2-hydroxy-3-methacryloxypropyl sulfonic acid, 2-acrylamido-2-methylpropane sulfonic acid, vinylphosphonic acid, allylphosphonic acid, and mixtures thereof.

Preferred, but nonlimiting, monomers include acrylic acid, methacrylic acid, maleic acid, fumaric acid, maleic anhydride, and the sodium, potassium, and ammonium salts thereof. An especially preferred monomer is acrylic acid.

The base polymer can contain additional monoethylenically unsaturated monomers that do not bear a pendant acid group, but are copolymerizable with monomers bearing acid groups. Such compounds include, for example, the amides and nitrites of monoethylenically unsaturated carboxylic acids, for example, acrylamide, methacrylamide, acrylonitrile, and methacrylonitrile. Examples of other suitable comonomers include, but are not limited to, vinyl esters of saturated C₁₋₄ carboxylic acids, such as vinyl formate, vinyl acetate, and vinyl propionate; alkyl vinyl ethers having at least two carbon atoms in the alkyl group, for example, ethyl vinyl ether and butyl vinyl ether; esters of monoethylenically unsaturated C₃₋₁₈ alcohols and acrylic acid, methacrylic acid, or maleic acid; monoesters of maleic acid, for example, methyl hydrogen maleate; acrylic and methacrylic esters of alkoxylated monohydric saturated alcohols, for example, alcohols having 10 to 25 carbon atoms reacted with 2 to 200 moles of ethylene oxide and/or propylene oxide per mole of alcohol; and monoacrylic esters and monomethacrylic esters of polyethylene glycol or polypropylene glycol, the molar masses (Mₙ) of the polyalkylene glycols being up to about 2,000, for example. Further suitable comonomers include, but are not limited to, styrene and alkyl-substituted styrenes, such as ethylstyrene and tert-butylstyrene, and 2-hydroxyethyl acrylate.

Polymerization of the acidic monomers, and any copolymerizable monomers, most commonly is performed by free radical processes in the presence of a polyfunctional organic compound. The base polymers are internally crosslinked to a sufficient extent such that the base polymer is water insoluble. Internal crosslinking renders the base polymer substantially water insoluble, and, in part, serves to determine the absorption capacity of the base polymer. For use in absorption applications, a base polymer is lightly crosslinked, i.e., has a crosslinking density of less than about 20%, preferably less than about 10%, and most preferably about 0.01% to about 7%.

A crosslinking agent most preferably is used in an amount of less than about 7 wt %, and typically about 0.1 wt % to about 5 wt %, based on the total weight of monomers. Examples of crosslinking polyvinyl monomers include, but are not limited to, polyacrylic (or polymethacrylic) acid esters represented by the following formula (I), and bisacrylamides represented by the following formula (II):

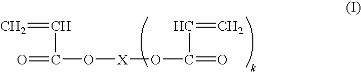

wherein X is ethylene, propylene, trimethylene, cyclohexyl, hexamethylene, 2-hydroxypropylene, —(CH₂CH₂O)ₙCH₂CH₂—, or

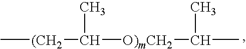

n and m are each an integer 5 to 40, and k is 1 or 2;

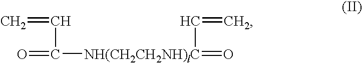

wherein 1 is 2 or 3.

The compounds of formula (I) are prepared by reacting polyols, such as ethylene glycol, propylene glycol, trimethylolpropane, 1,6-hexane-diol, glycerin, pentaerythritol, polyethylene glycol, or polypropylene glycol, with acrylic acid or methacrylic acid. The compounds of formula (II) are obtained by reacting polyalkylene polyamines, such as diethylenetriamine and triethylenetetramine, with acrylic acid.

Specific internal crosslinking agents include, but are not limited to, 1,4-butanediol diacrylate, 1,4-butanediol dimethacrylate, 1,3-butylene glycol diacrylate, 1,3-butylene glycol dimethacrylate, diethylene glycol diacrylate, diethylene glycol dimethacrylate, ethoxylated bisphenol A diacrylate, ethoxylated bisphenol A dimethacrylate, ethylene glycol dimethacrylate, 1,6-hexanediol diacrylate, 1,6-hexanediol dimethacrylate, neopentyl glycol dimethacrylate, polyethylene glycol diacrylate, polyethylene glycol dimethacrylate, triethylene glycol diacrylate, triethylene glycol dimethacrylate, tripropylene glycol diacrylate, tetraethylene glycol diacrylate, tetraethylene glycol dimethacrylate, dipentaerythritol pentaacrylate, pentaerythritol tetraacrylate, pentaerythritol triacrylate, trimethylolpropane triacrylate, trimethylolpropane trimethacrylate, tris(2-hydroxyethyl)-isocyanurate triacrylate, ethoxylated trimethylolpropane triacrylate (ETMPTA), e.g., ETMPTA ethyoxylated with 15 moles of ethylene oxide (EO) on average, tris(2-hydroxyethyl)isocyanurate trimethyacrylate, divinyl esters of a polycarboxylic acid, diallyl esters of a polycarboxylic acid, triallyl terephthalate, diallyl maleate, diallyl fumarate, hexamethylenebismaleimide, trivinyl trimellitate, divinyl adipate, diallyl succinate, a divinyl ether of ethylene glycol, cyclopentadiene diacrylate, a tetraallyl ammonium halide, divinyl benzene, divinyl ether, diallyl phthalate, or mixtures thereof. Especially preferred internal crosslinking agents are N,N′-methylenebisacrylamide, N,N′-methylenebismethacrylamide, ethylene glycol dimethacrylate, and trimethylolpropane triacrylate.

The base polymer can be any internally crosslinked polymer having pendant acid moieties that acts as an SAP in its neutralized form. Examples of base polymers include, but are not limited to, polyacrylic acid, hydrolyzed starch-acrylonitrile graft copolymers, starch-acrylic acid graft copolymers, saponified vinyl acetate-acrylic ester copolymers, hydrolyzed acrylonitrile copolymers, hydrolyzed acrylamide copolymers, ethylene-maleic anhydride copolymers, isobutylene-maleic anhydride copolymers, poly(vinylsulfonic acid), poly(vinylphosphonic acid), poly(vinylphosphoric acid), poly-(vinylsulfuric acid), sulfonated polystyrene, poly-(aspartic acid), poly(lactic acid), and mixtures thereof. The preferred base polymer is a homopolymer or copolymer of acrylic acid or methacrylic acid.

The free radical polymerization is initiated by an initiator or by electron beams acting on a polymerizable aqueous mixture. Polymerization also can be initiated in the absence of such initiators by the action of high energy radiation in the presence of photoinitiators.

Useful polymerization initiators include, but are not limited to, compounds that decompose into free radicals under polymerization conditions, for example, peroxides, hydroperoxides, persulfates, azo compounds, and redox catalysts. Water-soluble initiators are preferred. In some cases, mixtures of different polymerization initiators are used, for example, mixtures of hydrogen peroxide and sodium peroxodisulfate or potassium peroxodisulfate. Mixtures of hydrogen peroxide and sodium peroxodisulfate can be in any proportion.

Examples of suitable organic peroxides include, but are not limited to, acetylacetone peroxide, methyl ethyl ketone peroxide, tert-butyl hydroperoxide, cumeme hydroperoxide, tert-amyl perpivalate, tert-butyl perpivalate, tert-butyl perneohexanoate, tert-butyl perisobutyrate, tert-butyl per-2-ethylhexanoate, tert-butyl perisononanoate, tert-butyl permaleate, tert-butyl perbenzoate, di(2-ethylhexyl) peroxydicarbonate, dicyclohexyl peroxydicarbonate, di(4-tert-butylcyclohexyl) peroxydicarbonate, dimyristyl peroxydicarbonate, diacetyl peroxydicarbonate, an allyl perester, cumyl peroxyneodecanoate, tert-butyl per-3,5,5-trimethylhexanoate, acetylcyclohexylsulfonyl peroxide, dilauryl peroxide, dibenzoyl peroxide, and tert-amyl perneodecanoate. Particularly suitable polymerization initiators are water-soluble azo initiators, e.g., 2,2′-azobis(2-amidinopropane) dihydrochloride, 2,2′-azobis(N,N′-dimethylene)isobutyramidine dihydrochloride, 2-(carbamoylazo-isobutyronitrile, 2,2′-azobis-[2-(2′-imidazolin-2-yl)propane] dihydrochloride, and 4,4′-azobis(4-cyanovaleric acid). The polymerization initiators are used, for example, in amounts of 0.01% to 5%, and preferably 0.05% to 2.0%, by weight, based on the monomers to be polymerized.

Polymerization initiators also include redox catalysts. In redox catalysts, the oxidizing compound comprises at least one of the above-specified per compounds, and the reducing component comprises, for example, ascorbic acid, glucose, sorbose, ammonium or alkali metal bisulfite, sulfite, thiosulfate, hyposulfite, pyrosulfite, or sulfide, or a metal salt, such as iron (II) ions or sodium hydroxymethylsulfoxylate. The reducing component of the redox catalyst preferably is ascorbic acid or sodium sulfite. Based on the amount of monomers used in the polymerization, about 3×10⁻⁶ to about 1 mol % of the reducing component of the redox catalyst system can be used, and about 0.001 to about 5.0 mol % of the oxidizing component of the redox catalyst can be used, for example.

When polymerization is initiated using high energy radiation, the initiator typically comprises a photoinitiator. Photoinitiators include, for example, α-splitters, H-abstracting systems, and azides. Examples of such initiators include, but are not limited to, benzophenone derivatives, such as Michler's ketone; phenanthrene derivatives; fluorene derivatives; anthraquinone derivatives; thioxanthone derivatives; coumarin derivatives; benzoin ethers and derivatives thereof; azo compounds, such as the above-mentioned free-radical formers, substituted hexaarylbisimidazoles, acylphosphine oxides; or mixtures thereof.

Examples of azides include, but are not limited to, 2-(N,N-dimethylamino)ethyl 4-azido-cinnamate, 2-(N,N-dimethylamino)ethyl 4-azido-naphthyl ketone, 2-(N,N-dimethylamino)ethyl 4-azido-benzoate, 5-azido-1-naphthyl 2′-(N,N-dimethylamino)-ethyl sulfone, N-(4-sulfonylazidophenyl)maleimide, N-acetyl-4-sulfonylazidoaniline, 4-sulfonyl-azido-aniline, 4-azidoaniline, 4-azidophenacyl bromide, p-azidobenzoic acid, 2,6-bis(p-azidobenzylidene)cyclohexanone, and 2,6-bis(p-azidobenzylidene)-4-methyl-cyclohexanone. Photoinitiators customarily are used, if at all, in amounts of about 0.01% to about 5%, by weight of the monomers to be polymerized.

As previously stated, the base polymer is partially neutralized. The degree of neutralization is about 25 to about 100, preferably about 50 to about 90, mol %, based on monomers containing acid groups. The degree of neutralization more preferably is greater than about 60 mol %, even more preferably about 65 to about 90 mol %, most preferably about 65 to about 80 mol %, based on monomers containing acid groups.

Useful neutralizing agents for the base polymer include alkali metal bases, ammonia, and/or amines. Preferably, the neutralizing agent comprises aqueous sodium hydroxide, aqueous potassium hydroxide, or lithium hydroxide. However, neutralization also can be achieved using sodium carbonate, sodium bicarbonate, potassium carbonate, or potassium bicarbonate, or other carbonates or bicarbonates, as a solid or as a solution. Primary, secondary, and/or tertiary amines can be used to neutralize the base polymer.

Neutralization of the base polymer can be performed before, during, or after the polymerization in a suitable apparatus for this purpose. The neutralization is performed, for example, directly in a kneader used for polymerization of the monomers.

In accordance with the present invention, polymerization of an aqueous monomer solution, i.e., gel polymerization, is preferred. In this method, a 10% to 70%, by weight, aqueous solution of the monomers, including the internal crosslinking agent, is neutralized in the presence of a free radical initiator. The solution polymerization is performed at 0° C. to 150° C., preferably at 10° C. to 100° C., and at atmospheric, superatmospheric, or reduced pressure. The polymerization also can be conducted under a protective gas atmosphere, preferably under nitrogen.

After polymerization, the resulting hydrogel of the base polymer is dried, and the dry base polymer particles are ground and classified to a predetermined size for an optimum fluid absorption profile. In accordance with the present invention, the base polymer particles then are surface crosslinked. It should be understood that the polyamine coating process step and surface crosslinking process step are different, and impart different properties to the surfaces of the base polymer particles. The base polymer particles are surface crosslinked prior to application of the polyamine coating.

In one embodiment of applying a polyamine coating to the surface-crosslinked polymer particles, a surface-crosslinking agent is applied to the surfaces of the base polymer particles. Then, the resulting polymer particles are heated for a sufficient time and at a sufficient temperature to surface crosslink the base polymer particles. Next, a coating solution containing a polyamine dissolved in water and an optional cosolvent, and further containing an optional crosslinking agent, is applied to the surfaces of the surface-crosslinked SAP particles. The polyamine coating is applied to surface-crosslinked SAP particles having a temperature of about 25° C. to about 100° C., and preferably about 50° C. to about 100° C. The polyamine coating is added to surface-crosslinked SAP particles after the surface-crosslinking step, wherein the surface-cross-linked SAP particles are cooling, but still warm. Accordingly, the polyamine coating is applied using the latent heat of the surface-crosslinked SAP particles. If needed, an external heat source can be used to achieve a desired polyamine-coated SAP particle temperature of up to about 100° C.

After applying the polyamine coating to the surface-crosslinked SAP particles, the coated SAP particles are mixed for about 5 to about 60 minutes to form a uniform polyamine coating on the surface-crosslinked polymer particles and provide SAP particles of the present invention. The polyamine coating is hydrophilic in the absence of an optional cosolvent, and is hydrophobic in the presence of an optional cosolvent.

The components of the polyamine coating solution can be applied to the SAP particles in any order, from one, two, or three solutions. In particular, the cosolvent and optional crosslinking agent can be applied to the surface-crosslinked SAP particles independent of the polyamine and independent of each other. Alternatively, the polyamine, optional cosolvent, and optional crosslinking agent can be administered and applied from a single solution.

In the surface crosslinking process, a multifunctional compound capable of reacting with the functional groups of the base polymer is applied to the surface of the base polymer particles, preferably using an aqueous solution. The aqueous solution also can contain water-miscible organic solvents, like an alcohol, such as methanol, ethanol, or i-propanol; a polyol, like ethylene glycol or propylene glycol; or acetone.

A solution of a surface-crosslinking agent is applied to the base polymer particles in an amount to wet predominantly only the outer surfaces of the base polymer particles, either before or after application of the polyamine. Surface cross-linking and drying of the base polymer particles then is performed, preferably by heating at least the wetted surfaces of the base polymer particles.

Typically, the base polymer particles are surface treated with a solution of a surface-cross-linking agent containing about 0.01% to about 4%, by weight, surface-crosslinking agent, and preferably about 0.4% to about 2%, by weight, surface-cross-linking agent in a suitable solvent. The solution can be applied as a fine spray onto the surfaces of freely tumbling base polymer particles at a ratio of about 1:0.01 to about 1:0.5 parts by weight base polymer particles to solution of surface-crosslinking agent. The surface-crosslinking agent is present in an amount of 0.001% to about 5%, by weight of the base polymer particles, and preferably 0.001% to about 0.5% by weight. To achieve the full advantage of the present invention, the surface-crosslinking agent is present in an amount of about 0.001% to about 0.2%, by weight of the base polymer particles.

Surface crosslinking of the base polymer particles and drying are achieved by heating the surface-treated base polymer particles at a suitable temperature, e.g., about 70° C. to about 200° C., and preferably about 105° C. to about 180° C. Suitable surface-crosslinking agents are capable of reacting with acid moieties and crosslinking polymers at the surfaces of the base polymer particles.

Nonlimiting examples of suitable surface-crosslinking agents include, but are not limited to, an alkylene carbonate, such as ethylene carbonate or propylene carbonate; a polyaziridine, such as 2,2-bishydroxymethyl butanol tris[3-(1-aziridine propionate] or bis-N-aziridinomethane; a haloepoxy, such as epichlorohydrin; a polylsocyanate, such as 2,4-toluene diisocyanate; a di- or polyglycidyl compound, such as diglycidyl phosphonates, ethylene glycol diglycidyl ether, or bischlorohydrin ethers of polyalkylene glycols; alkoxysilyl compounds; polyols such as ethylene glycol, 1,2-propanediol, 1,4-butanediol, glycerol, methyltriglycol, polyethylene glycols having an average molecular weight M_{w} of 200-10,000, di- and polyglycerol, pentaerythritol, sorbitol, the ethoxylates of these polyols and their esters with carboxylic acids or carbonic acid, such as ethylene carbonate or propylene carbonate; carbonic acid derivatives, such as urea, thiourea, guanidine, dicyandiamide, 2-oxazolidinone and its derivatives, bisoxazoline, polyoxazolines, di- and polyisocyanates; di- and poly-N-methylol compounds, such as methylenebis(N-methylolmethacrylamide) or melamine-formaldehyde resins; compounds having two or more blocked isocyanate groups, such as trimethylhexamethylene diisocyanate blocked with 2,2,3,6-tetramethylpiperidin-4-one; 2-hydroxyethyloxazolidinone; hydroxyalkylamides as disclosed in U.S. Pat. No. 6,239,230, incorporated herein by reference; and other surface-crosslinking agents known to persons skilled in the art.

A polyamine is applied to the polymer particles after the surface crosslinking step has been completed. A solution containing the polyamine comprises about 5% to about 50%, by weight, of a polyamine in a suitable solvent. Typically, a sufficient amount of a solvent is present to allow the polyamine to be readily and homogeneously applied to the surfaces of the base polymer particles. The solvent for the polyamine solution typically comprises water.

The amount of polyamine applied to the surfaces of the surface-crosslinked polymer particles is sufficient to coat the surface-crosslinked polymer particle surfaces. Accordingly, the amount of polyamine applied to the surfaces of the surface-crosslinked polymer particles is about 0.1% to about 2%, and preferably about 0.2% to about 1%, of the weight of the surface-crosslinked polymer particles. To achieve the full advantage of the present invention, the polyamine is present on the surface-cross-linked polymer particle surfaces in an amount of about 0.2% to about 0.5%, by weight of the surface-crosslinked polymer particles.

A polyamine can form an ionic bond with a surface-crosslinked polymer particle and retains adhesive forces to the surface-crosslinked particle after the surface-crosslinked polymer absorbs a fluid and swells. Preferably, an excessive amount of covalent bonds are not formed between the polyamine and the surface-crosslinked polymer particle, and the polyamine surface-crosslinked polymer particle interactions are intermolecular, such as electrostatic, hydrogen bonding, and van der Waals interactions. Therefore, the presence of a polyamine on the surface-crosslinked polymer particles does not adversely influence the absorption profile of the surface-crosslinked polymer particles.

A polyamine useful in the present invention has at least two, and preferably a plurality, of nitrogen atoms per molecule. The polyamine typically has a weight average molecular weight (M_{w}) of about 5,000 to about 1,000,000, and preferably about 20,000 to about 600,000. To achieve the full advantage of the present invention, the polyamine has an M_{w} of about 100,000 to about 400,000.

In general, useful polyamines have (a) primary amino groups, (b) secondary amino groups, (c) tertiary amino groups, (d) quaternary ammonium groups, or (e) mixtures thereof. Examples of polyamines include, but are not limited to, a polyvinylamine, a polyallylamine, a polyethyleneimine, a polyalkyleneamine, a polyazetidine, a polyvinylguanidine, a poly(DADMAC), i.e., a poly(diallyl dimethyl ammonium chloride), a cationic polyacrylamide, a polyamine functionalized polyacrylate, and mixtures thereof.

Homopolymers and copolymers of vinylamine also can be used, for example, copolymers of vinylformamide and comonomers, which are converted to vinylamine copolymers. The comonomers can be any monomer capable of copolymerizing with vinylformamide. Nonlimiting examples of such monomers include, but are not limited to, acrylamide, methacrylamide, methacrylonitrile, vinylacetate, vinylpropionate, styrene, ethylene, propylene, N-vinylpyrrolidone, N-vinylcaprolactam, N-vinylimidazole, monomers containing a sulfonate or phosphonate group, vinylglycol, acrylamido(methacrylamido)alkylene trialkyl ammonium salt, diallyl dialkylammonium salt, C₁₋₄alkyl vinyl ethers such as methyl vinyl ether, ethyl vinyl ether, isopropyl vinyl ether, n-propyl vinyl ether, t-butyl vinyl ether, N-substituted alkyl (meth)acrylamides substituted by a C₁₋₄alkyl group as, for example, N-methylacrylamide, N-isopropylacrylamide, and N,N-dimethylacrylamide, C₁₋₂₀alkyl(meth)acrylic acid esters such as methyl methacrylate, ethyl methacrylate, propyl acrylate, butyl acrylate, hydroxyethyl acrylate, hydroxyethyl methacrylate, hydroxypropyl acrylate, hydroxypropyl methacrylate, hydroxybutyl acrylate, hydroxybutyl methacrylate, 2-methylbutyl acrylate, 3-methylbutyl acrylate, 3-pentyl acrylate, neopentyl acrylate, 2-methylpentyl acrylate, hexyl acrylate, cyclohexyl acrylate, 2-ethylhexyl acrylate, phenyl acrylate, heptyl acrylate, benzyl acrylate, tolyl acrylate, octyl acrylate, 2-octyl acrylate, nonyl acrylate, and octyl methacrylate.

Specific copolymers of polyvinylamine include, but are not limited to, copolymers of N-vinylformamide and vinyl acetate, vinyl propionate, a C₁₋₄alkyl vinyl ether, a (meth)acrylic acid ester, acrylonitrile, acrylamide, or vinylpyrrolidone.

A polyamine coating is hydrophilic as applied to the surface-crosslinked polymer particles. The polyamine coating can be rendered hydrophobic by including a cosolvent in the polyamine coating process. The optional cosolvent contains at least one, and often two or three, hydroxy groups. Useful cosolvents include, but are not limited to, alcohols, diols, triols, and mixtures thereof, for example, methanol, ethanol, propyl alcohol, isopropyl alcohol, ethylene glycol, propylene glycol, oligomers of ethylene glycol, oligomers of propylene glycol, glycerin, monoalkyl ethers of propylene glycol, and similar hydroxy-containing solvents. An oligomer of ethylene glycol or propylene glycol contains two to four ethylene oxide or propylene oxide monomer units.

In accordance with the present invention, the number of covalent bonds that form between the polyamine and the surface-crosslinked SAP particles is low, if present at all. A polyamine alone may impart a tack to surfaces of the base polymer particles, which leads to agglomeration or aggregation of coated base polymer particles especially if the polyamine coating is hydrophilic. To overcome this potential problem, a crosslinking agent for a polyamine coating can be used.

Crosslinking of the polyamine coating is different from surface crosslinking. The crosslinking agent for the polyamine coating forms crosslinks between the nitrogen atoms of the polyamine. The surface crosslinking agent forms crosslinks with carboxyl groups of the base polymer. In addition, the surface crosslinking agent is applied to the base polymer and reacted prior to application of the polyamine coating. However, it should be understood that the crosslinking agent for the polyamine coating in some embodiments may react with the nitrogen atoms of the polyamine and a small number of carboxyl groups of the base polymer.

The crosslinking agent for the polyamine coating can be organic or inorganic in nature. An organic crosslinking agent reacts with nitrogen atoms of the polyamine to form covalent bonds with the polyamine nitrogen atoms. An inorganic cross-linking agent forms ionic crosslinks via the nitrogen atoms of the polyamine coating. The crosslinking agents can be used individually or in admixture, e.g., a mixture of inorganic crosslinking agents, a mixture of organic crosslinking agents, or a mixture of inorganic and organic crosslinking agents.

In one embodiment, the crosslinking agent is a solution containing a salt having (a) a polyvalent metal cation, i.e., a metal cation having a valence of two, three, or four, (b) a polyvalent anion, i.e., an anion having a valence of two or greater, or (c) both a polyvalent cation and a polyvalent anion, is applied to the surfaces of the surface-crosslinked polymer particles. In this embodiment, the salt is applied to the surface-crosslinked polymer particles independently from the polyamine in order to avoid a premature crosslinking reaction. The salt can be applied to the surface-crosslinked polymer particles prior to or after the polyamine is added to the surface of the surface-crosslinked polymer particles.

The polyvalent metal cation and polyvalent anion are capable of interacting, e.g., forming ionic crosslinks, with the nitrogen atoms of the polyamine. As a result, a tackless polyamine coating is formed on the surface of the base polymer to provide coated SAP particles of the present invention.

In accordance with the present invention, a salt applied to surfaces of the base polymer particles has a sufficient water solubility such that polyvalent metal cations and/or polyvalent anions are available to interact with the nitrogen atoms of the polyamine. Accordingly, a useful salt has a water solubility of at least 0.01 g of salt per 100 ml of water, and preferably at least 0.02 g per 100 ml of water.

A polyvalent metal cation of the salt has a valence of +2, +3, or +4, and can be, but is not limited to, Mg²⁺, Ca²⁺, Al³⁺, Sc³⁺, Ti⁴⁺, Mn²⁺, Fe^{2+/3+}, Co²⁺, Ni²⁺, Cu^{+/2+}, Zn²⁺, Y³⁺, Zr⁴⁺, La³⁺, Ce⁴⁺, Hf⁴⁺, Au³⁺, and mixtures thereof. Preferred cations are Mg²⁺, Ca²⁺, Al³⁺, Ti⁴⁺, Zr⁴⁺, La³⁺, and mixtures thereof, and particularly preferred cations are Al³⁺, Ti⁴⁺, Zr⁴⁺, and mixtures thereof. The anion of a salt having a polyvalent cation is not limited, as long as the salt has sufficient solubility in water. Examples of anions include, but are not limited to, chloride, bromide, and nitrate.

A polyvalent anion of the salt has a valence of −2, −3, or −4. The polyvalent anion can be inorganic or organic in chemical structure. The identity of the polyvalent anion is not limited as long as the anion is capable of interacting with the nitrogen atoms of the polyamine.

Examples of polyvalent inorganic anions include, but are not limited to, sulfate, phosphate, hydrogen diphosphate, and borate. Examples of polyvalent organic anions include, but are not limited to, water-soluble anions of polycarboxylic acids. In particular, the anion can be an anion of a di- or tri-carboxylic acid, such as oxalic acid, tartaric acid, lactic acid, malic acid, citric acid, aspartic acid, malonic acid, and similar water-soluble polycarboxylic acids optionally containing a hydroxy and/or an amino group. Additional useful polyvalent anions include polycarboxylic amino compounds, for example, polyacrylic acid, ethylenediaminetetraacetic acid (EDTA), ethylenebis(oxyethylenenitrile)-tetraacetic acid (EGTA), diethylenetriaminopentaacetic acid (DTPA), N-hydroxyethylethylenedlaminetriacetic acid (HEDTA), and mixtures thereof.

In addition, a salt containing a polyvalent metal cation and a polyvalent anion can be used, provided the salt has sufficient water solubility to be dissolved in a solvent for a homogeneous application to surface-crosslinked SAP particles.

The salt can be present in a coating solution together with an optional organic crosslinking agent. The salt typically is present in the coating solution in an amount of about 0.5% to 20%, by weight, for example. The amount of salt present in a coating solution, and the amount applied to the surface-crosslinked polymer particles, is related to the identity of the salt, its solubility in the solvent of the coating solution, the identity of the polyamine applied to the surface-crosslinked polymer particles, and the amount of polyamine applied to the surface-crosslinked polymer particles. In general, the amount of salt applied to the surface-crosslinked polymer particles is sufficient to form a tackless, monolithic polyamine coating and provide coated SAP particles of the present invention.

In another embodiment, an organic cross-linking agent can be used in conjunction with the polyamine. In still another embodiment, an organic crosslinking agent is applied to the surface cross-linked polymer particles, followed by the polyamine solution. The optional cosolvent can be applied to the surface-crosslinked polymer particles with the organic crosslinking agent, with the polyamine, with both, or alone, either before or after application of the organic crosslinking agent or the polyamine. In either case, the SAP particles then are maintained at a sufficient temperature for a sufficient time to form crosslinks between the polyamine and the crosslinking agent.

In the organic crosslinking process, a multifunctional compound capable of reacting with the amino groups of the polyamine is applied to the surface of the surface-crosslinked polymer particles. The organic crosslinking agent can be the same or different from the surface crosslinking agent. However, as discussed above, the surface crosslinking agent and the crosslinking agent for the polyamine are applied to the base polymer particles during different process steps and the SAP particles are maintained at different temperatures, i.e., the surface crosslinking process utilizes a higher temperature to effect a reaction with the carboxy groups of the base polymer, and the polyamine crosslinking process utilizes a lower temperature for crosslinking through the nitrogen atoms of the polyamine.

The organic crosslinking process typically utilizes an aqueous solution of the crosslinking agent. The aqueous solution also can contain water-miscible organic solvents, like an alcohol, such as methanol, ethanol, or i-propanol; a polyol, like ethylene glycol or propylene glycol; or acetone.

A solution of an organic crosslinking agent is applied to the surface-crosslinked polymer particles during or after application of the polyamine in an amount to wet predominantly only the outer surfaces of the surface-crosslinked polymer particles. Crosslinking and drying of the coated surface-crosslinked polymer particles then are achieved by maintaining at least the wetted surfaces of the surface-crosslinked polymer particles at a suitable temperature, e.g., about 25° C. to about 100° C., preferably about 50° C. to about 100° C., and more preferably about 60° C. to about 90° C., for about 5 to about 60 minutes to allow the crosslinking agent to react with the nitrogen atoms of the polyamine.

Typically, the surface-crosslinked polymer particles are treated with a solution of an organic crosslinking agent containing about 0.5% to about 20%, by weight, crosslinking agent, and preferably about 3% to about 15%, by weight, crosslinking agent in a suitable solvent. The organic crosslinking agent, if present at all, is present in an amount of 0.001% to about 0.5%, by weight of the surface-crosslinked polymer particles, and preferably 0.001% to about 0.3% by weight. To achieve the full advantage of the present invention, the organic cross-linking agent is present in an amount of about 0.001% to about 0.1%, by weight of the surface-crosslinked polymer particles.

Nonlimiting examples of suitable organic crosslinking agents include, but are not limited to, an alkylene carbonate, such as ethylene carbonate or propylene carbonate; a polyaziridine, such as 2,2-bishydroxymethyl butanol tris[3-(1-aziridine propionate] or bis-N-aziridinomethane; a haloepoxy, such as epichlorohydrin; a polyisocyanate, such as 2,4-toluene diisocyanate; a di- or polyglycidyl compound, such as diglycidyl phosphonates, ethylene glycol diglycidyl ether, or bischlorohydrin ethers of polyalkylene glycols; alkoxysilyl compounds; carbonic acid derivatives, such as urea, thiourea, guanidine, dicyandiamide, 2-oxazolidinone and its derivatives, bisoxazoline, polyoxazolines, di- and polyisocyanates; di- and poly-N-methylol compounds, such as methylenebis(N-methylolmethacrylamide) or melamine-formaldehyde resins; compounds having two or more blocked isocyanate groups, such as trimethylhexamethylene diisocyanate blocked with 2,2,3,6-tetramethylpiperidin-4-one; multifunctional aldehydes, multifunctional ketones, multifunctional acetals, multifunctional ketals, and other organic crosslinking agents known to persons skilled in the art. The organic crosslinking agent can be used alone or in combination.

A solution of the organic crosslinking agent is applied to the surfaces of the surface-crosslinked polymer particles simultaneously with, or before or after, a solution containing the polyamine is applied to the surfaces of the surface-crosslinked polymer particles. The polyamine is applied to the particles after a surface crosslinking step has been completed.

In accordance with the present invention, the polyamine solution, and inorganic and/or organic crosslinking agent, are applied to the surface-crosslinked polymer particles in a manner such that each is uniformly distributed on the surfaces of the surface-crosslinked polymer particles. In addition to the crosslinking agent, other optional ingredients can be applied to the surface crosslinked SAP particles in conjunction with the polyamine. Such optional ingredients include, but are not limited to, clay and silica, for example, to impart anticaking properties to the polyamine-coated SAP particles. A clay or silica also can be added to the polyamine-coated SAP particles after application and curing of the polyamine coating.

Any known method for applying a liquid to a solid can be used to apply the polyamine coating to the surface-crosslinked SAP particles, preferably by dispersing a coating solution into fine droplets, for example, by use of a pressurized nozzle or a rotating disc. Uniform coating of the surface-crosslinked polymer particles can be achieved in a high intensity mechanical mixer or a fluidized mixer which suspends the surface-crosslinked polymer particles in a turbulent gas stream. Methods for the dispersion of a liquid onto the surfaces of surface-crosslinked polymer particles are known in the art, see, for example, U.S. Pat. No. 4,734,478, incorporated herein by reference.

Methods of coating the surface-crosslinked polymer particles include applying the polyamine and crosslinking agent simultaneously. When an inorganic salt is used as a crosslinking agent, the polyamine and salt preferably are applied via two separate nozzles to avoid an interaction before application to the surfaces of the surface-cross-linked polymer particles. A preferred method of coating the surface-crosslinked polymer is a sequential addition of the components. A more preferred method is a first application of the polyamine, followed by an application of the crosslinking agent.

The resulting coated surface-crosslinked polymer particles then are maintained at about 25° C. to about 100° C. for sufficient time, e.g., about 5 to about 60 minutes. In particular, the polyamine coating typically is applied to surface-crosslinked SAP particles that have not completely cooled after the surface-crosslinking process. Accordingly, the polyamine-coating step utilizes the latent heat of the surface-crosslinked SAP particles. If necessary, external heat can be applied to maintain a desired particle temperature up to about 100° C. and cure the polyamine coating. The temperature of the polyamine-coated SAP particles is maintained at about 100° C. or less to avoid, or at least minimize, reactions that form covalent bonds between the polyamine coating and the carboxyl groups of the base polymer.

After application of the polyamine, water, optional solvent, and optional crosslinking agent to the surface-crosslinked SAP particles, the coated SAP particles are mixed at about 25° C. to about 100° C., e.g., 25, 30, 35, 40, 45, 50, 55, 60, 65, 70, 75, 80, 85, 90, 95, or 100° C., for about 5 to about 60 minutes in a paddle mixer, for example, such as those available from Ruberg-Mischtechnik AG, Nieheim, Germany and Nara Machining Co., Ltd., Frechen, Germany. Other suitable mixers include Patterson-Kelly mixers, DPAIS turbulence mixers, Lödige mixers, Schugi mixers, screw mixers, and pan mixers. After mixing, a polyamine coated SAP of the present invention results, i.e., a surface-cross-linked SAP particle having an optionally crosslinked polyamine coating, wherein covalent bonds between the polyamine and the carboxyl groups of the base polymer are minimized.

The polyamine-coated SAP particles of the present invention have excellent absorption properties, permeability, and gel integrity. In particular, the present SAP particles have a centrifuge retention capacity of at least 25 g/g. The present particles also exhibit a gel integrity of at least 2, preferably at least 2.5, still more preferably at least 3, yet more preferably at least 3.5, and most preferably at least 4.0. The present SAP particles further exhibit a free swell gel bed permeability of at least 200, preferably at least 210, 220, 230, 240, or 250, and more preferably 260, 270, 280, 290, or 300 Darcies and preferably a gel bed permeability (0.3 psi) of at least 3, more preferably at least 4, 5, 6, or 7, and most preferably at least 8, 9, or 10 Darcies.

The present invention, therefore, provides polyamine-coated SAP particles having improved absorbency, fluid permeability, and gel integrity. Surprisingly, the absorbency, fluid permeability, and gel integrity properties are independent of wicking index, i.e., as wicking index decrease, the expected decrease in the absorbency and permeability properties are not observed.

The present invention also provides polyamine-coated SAP particles that have a hydrophobic surface when a cosolvent is applied as a component of the coating solution, which reduces SAP particle agglomeration attributed to the viscous, tacky nature of polyamines. The present invention also provides polyamine-coated SAP particles having a hydrophilic surface when an inorganic or organic crosslinking agent is applied as a component of the coating solution, and the SAP particles are maintained at a relatively low temperature, i.e., about 25° C. to about 100° C., preferably about 50° C. to about 100° C., and most preferably about 60° C. to about 80° C., for about 5 to about 60 minutes.

In accordance with the present invention, a polyamine is applied to surface-crosslinked SAP particles in a manner such that the polyamine and any optional crosslinking agent are uniformly distributed on the surfaces of the surface-crosslinked SAP particles. The resulting coated surface-cross-linked SAP particles then are maintained at about 25° C. to about 100° C., preferably about 50° C. to about 100° C., and more preferably about 60° C. to about 80° C., for sufficient time, e.g., about 5 to about 60, and preferably about 10 to about 30 minutes, to cross-link the polyamine coating, while minimizing covalent crosslinks between the polyamine coating and the carboxyl groups of the base polymer.

To demonstrate the unexpected advantages provided by the coated SAP particles of the present invention, polyamine-coated SAP particles were prepared and tested for centrifuge retention capacity (CRC, g/g), absorbency under load (AUL 0.9 psi, g/g), free swell gel bed permeability (GBP, Darcies), gel bed permeability (GBP 0.3 psi, Darcies), gel integrity (GI) (scale of 1 to 4), and fluid wicking index (cm/min). These tests were performed using the following procedures.

### Centrifuge Retention Capacity (CRC)

This test determines the free swelling capacity of a hydrogel-forming polymer. The resultant retention capacity is stated as grams of liquid retained per gram weight of the sample (g/g). In this method, 0.2000±0.0050 g of dry SAP particles of size fraction 106 to 850 μm are inserted into a teabag. A heat-sealable teabag material, such as that available from Dexter Corporation (having a place of business in Windsor Locks, Conn., U.S.A.) as model designation 1234T heat-sealable filter paper works well for most applications. The bag is formed by folding a 5-inch by 3-inch sample of the bag material in half and heat sealing two of the open edges to form a 2.5-inch by 3-inch rectangular pouch. The heat seals should be about 0.25 inches inside the edge of the material. After the sample is placed in the pouch, the remaining open edge of the pouch also is heat sealed. Empty bags also can be made to serve as controls. The teabag is placed in saline solution (i.e., 0.9 wt % aqueous sodium chloride) for 30 minutes (at least 0.831 (liter) saline solution/1 g polymer), making sure that the bags are held down until they are completely wetted. Then, the teabag is centrifuged for three minutes at 250 G. The absorbed quantity of saline solution is determined by measuring the weight of the teabag. The amount of solution retained by the superabsorbent polymer sample, taking into account the solution retained by the bag itself, is the centrifuge retention capacity (CRC) of the superabsorbent polymer, expressed as grams of fluid per gram of superabsorbent polymer. More particularly, the retention capacity is determined by the following equation:

$$\frac{\begin{matrix}sample bag after centrifuge \text{-} empty \\ bag after centrifuge \text{-} dry sample weight\end{matrix}}{dry sample weight}$$

Gel Bed Permeability (GBP Free Swell and 0.3 psi, Darcies)

This procedure is identical to that disclosed in U.S. Patent Publication No. 2005/0256757, incorporated herein by reference. The method is modified by using a 100 gram weight to provide 0.3 psi.

### Absorbency Under Load (AUL)

This procedure is disclosed in WO 00/62825, pages 22-23, incorporated herein by reference, using a 317 gram weight for an AUL (0.90 psi).

### Gel Integrity (GI)

In a small polystyrene weighing dish (one inch diameter base) place a 1 g of an SAP sample. The SAP particles are evenly distributed on the bottom of the dish, then 1 g of 0.9 wt % saline is added to the center of the SAP particles. The particles are allowed to stand for one minute and then evaluated:

| Properties | Grade |
|---|---|
| Loose particles, unable to pick up as one mass. | 1 |
| Can lift as one mass with thumb and forefinger, but tears under | 2 |
| its own weight. | |
| Can lift as one mass with thumb and forefinger, but tears when | 3 |
| oscillated in the horizontal direction. | |
| Can lift as one mass with thumb and forefinger, but tears with | 4 |
| the use of the opposing thumb and forefinger. | |

### Fluid Wicking Index

This procedure is identical to that disclosed in European Patent No. EP 0 532 002 B1, incorporated herein by reference.

### Particle Size Distribution (PSD)

Particle size distribution is determined as set forth in U.S. Pat. No. 5,061,259, incorporated herein by reference. In summary, a sample of SAP particles is added to the top of a series of stacked sieves. The sieves are mechanically shaken for a predetermined time, then the amount of SAP particles on each sieve is weighed. The percent of SAP particles on each sieve is calculated from the initial sample weight of the SAP sample.

### Example 1

Surface-crosslinked polymer particles, HySorb B-8700AD available from BASF AG, Ludwigshafen, Germany, were preheated in a laboratory oven set at a predetermined coating temperature. When the polymer particles (1 kg) attained a predetermined coating temperature, the surface-crosslinked polymer particles were transferred to a preheated laboratory Lödige mixer. The particles were maintained at the constant predetermined temperature throughout the coating step. Addition of a polyvinylamine coating solution (i.e., 40 grams LUPAMIN® 9095 and 15 grams of deionized water) to the preheated polymer particles was performed by disposable syringe, dropwise over 5 minutes at a Lödige mixing speed of 449 rpm. After complete addition of the coating solution, the Lödige mixing speed was reduced to 79 rpm, and mixing was continued for 30 minutes.

| | | Propylene | | | |
|---|---|---|---|---|---|
| | LUPAMIN ®¹⁾ | glycol | | Coating | Residence |
| Sample | 9095 (wt %) | (PG) (wt %) | H₂O (wt %) | Temp (° C.) | Time (min.) |
| Control | Base polymer (HySorb B-8700AD) | | | | |
| 1a | 4.0 | 0.0 | 1.5 | 60 | 30 |
| 1b | 4.0 | 0.0 | 1.5 | 70 | 30 |
| 1c | 4.0 | 0.0 | 1.5 | 80 | 30 |

| | | | | | | Gel | |
|---|---|---|---|---|---|---|---|
| | PSD | | AUL | | | Integrity | |
| | (>860μ | CRC | 0.9 psi | FS GBP | GBP 0.3 psi | (1~4 | |
| | wt %) | (g/g) | (g/g) | (Darcies) | (Darcies) | scale) | SAP surface |
| Control | | 26.28 | 19.60 | 85.5 | 8.48 | 1.0 | Hydrophilic |
| 1a | 5.94 | 24.90 | 17.23 | 229.0 | 6.59 | 4.0 | Hydrophilic |
| 1b | 6.27 | 25.29 | 18.91 | 181.4 | 7.29 | 4.0 | Hydrophilic |
| 1c | 6.66 | 25.66 | 19.25 | 152.8 | 6.38 | 2.5 | Hydrophilic |

Wicking index (cm)

| | 1 min. | 5 min. | 10 min. |
|---|---|---|---|
| Control | 6.0 | 11.0 | 15.0 |
| 1a | 2.6 | 5.5 | 7.0 |
| 1b | 3.5 | 6.0 | 8.0 |
| 1c | 3.0 | 6.0 | 7.0 |

¹⁾LUPAMIN ® 9095, available from BASF Corporation, Florham Park, NJ, contains 5-10% linear polyvinylamine, average molecular weight 340,000.

### Example 2

Surface-crosslinked polymer particles, HySorb B-8700AD, were preheated in a laboratory oven set at a predetermined coating temperature. When the polymer particles (1 kg) attained a predetermined coating temperature, the particles were transferred to a preheated laboratory Lödige mixer. The polymer particles were maintained at the constant predetermined temperature throughout the coating step. Addition of a polyvinylamine coating solution (40 grams LUPAMIN® 9095, 10 grams propylene glycol (PG), and 15 grams of deionized (DI) water) to the preheated polymer particles was performed by disposable syringe, dropwise over 5 minutes at a Lödige mixing speed of 449 rpm. After complete addition of the coating solution, the Lödige mixing speed was reduced to 79 rpm, and mixing was continued for 30 minutes.

| | | Propylene | | | |
|---|---|---|---|---|---|
| | LUPAMIN ® | glycol (PG) | | Coating | Residence |
| Sample | 9095 (wt %) | (wt %) | H₂O (wt %) | Temp (° C.) | Time (min.) |
| Control | Base polymer (HySorb B-8700AD) | | | | |
| 2a | 4.0 | 1.0 | 1.5 | 60 | 30 |
| 2b | 4.0 | 1.0 | 1.5 | 70 | 30 |
| 2c | 4.0 | 1.0 | 1.5 | 80 | 30 |

| | | | | | | Gel | |
|---|---|---|---|---|---|---|---|
| | PSD | | AUL | | | Integrity | |
| | (>860μ | CRC | 0.9 psi | FS GBP | GBP 0.3 psi | (1~4 | |
| | wt %) | (g/g) | (g/g) | (Darcies) | (Darcies) | scale) | SAP surface |
| Control | | 26.28 | 19.60 | 85.5 | 8.48 | 1.0 | Hydrophilic |
| 2a | 0.22 | 24.59 | 17.45 | 342.5 | 2.45 | 4.0 | Hydrophobic |
| 2b | 0.24 | 25.14 | 17.04 | 356.1 | 3.16 | 4.0 | Hydrophobic |
| 2c | 0.34 | 25.69 | 17.39 | 303.6 | 3.34 | 3.5 | Hydrophobic |

Wicking index (cm)

| | 1 min. | 5 min. | 10 min. |
|---|---|---|---|
| Control | 6.0 | 11.0 | 15.0 |
| 2a | 0.4 | 1.0 | 1.5 |
| 2b | 0.4 | 1.0 | 1.5 |
| 2c | 0.4 | 1.0 | 2.0 |

### Example 3

Surface-crosslinked polymer particles, HySorb B-8700AD, were preheated in a laboratory oven set at a predetermined temperature. When the polymer particles (1 kg) attained a predetermined temperature, the particles were transferred to a preheated laboratory Lödige mixer. The polymer particles were maintained at the constant predetermined temperature throughout the coating step. Preparation of Solution 1: alum solution (35.8 grams, 28.1 wt % aluminum sulfate) in first disposable syringe, and Solution 2: polyvinylamine coating solution (40 or 20 grams LUPAMIN® 9095, 10 grams PG) in second disposable syringe. Solution 1 was added, first, then Solution 2, to the preheated polymer particles. The additions were performed dropwise over 5 minutes at a Lödige mixing speed of 449 rpm. After complete addition of the coating solutions, the Lödige mixing speed was reduced to 79 rpm, and mixing was continued for 30 minutes.

| | | Propylene | | | |
|---|---|---|---|---|---|
| | LUPAMIN ® | glycol (PG) | Aluminum | Coating | Residence |
| Sample | 9095 (wt %) | (wt %) | sulfate (wt %) | Temp (° C.) | Time (min.) |
| Control | Base polymer (HySorb B-8700AD) | | | | |
| 3a | 4.0 | 1.0 | — | 60 | 30 |
| 3b | 4.0 | 1.0 | 1 | 60 | 30 |
| 3c | 4.0 | 1.0 | 1 | 100 | 30 |
| 3d | 2.0 | 1.0 | — | 60 | 30 |
| 3e | 2.0 | 1.0 | 1 | 60 | 30 |
| 3f | 2.0 | 1.0 | 1 | 100 | 30 |

| | | | | | | Gel | |
|---|---|---|---|---|---|---|---|
| | PSD | | AUL | | | Integrity | |
| | (>860μ | CRC | 0.9 psi | FS GBP | GBP 0.3 psi | (1~4 | |
| | wt %) | (g/g) | (g/g) | (Darcies) | (Darcies) | scale) | SAP surface |
| Control | | 26.28 | 19.60 | 85.53 | 8.58 | 1.0 | Hydrophilic |
| 3a | 0.17 | 24.89 | 18.50 | 351.60 | 2.06 | 4.0 | Hydrophobic |
| 3b | 0.19 | 24.30 | 16.94 | 317.48 | 7.01 | 4.0 | Hydrophilic |
| 3c | 0.40 | 24.93 | 16.79 | 323.23 | 3.63 | 4.0 | Hydrophilic |
| 3d | 1.94 | 23.85 | 18.11 | 253.42 | 11.50 | 3.5 | Hydrophobic |
| 3e | 0.14 | 24.80 | 17.75 | 247.73 | 9.20 | 2.5 | Hydrophilic |
| 3f | 0.64 | 25.00 | 16.97 | 260.73 | 9.55 | 3.5 | Hydrophilic |

Wicking index (cm)

| | 1 min. | 5 min. | 10 min. |
|---|---|---|---|
| Control | 7.0 | 11.0 | 14.5 |
| 3a | 1.0 | 1.5 | 2.0 |
| 3b | 5.5 | 7.5 | 9.0 |
| 3c | 3.0 | 4.9 | 6.0 |
| 3d | 1.0 | 4.0 | 6.0 |
| 3e | 7.0 | 10.5 | 13.0 |
| 3f | 5.5 | 9.5 | 11.0 |

### Example 4

Surface-crosslinked polymer particles, HySorb B-8700AD, were preheated in a laboratory oven at 60° C. When the polymer particles (1 kg) reached 60° C., the particles were transferred to a preheated (60° C.) laboratory Lödige mixer. The polymer particles were maintained at 60° C. throughout the coating step. Preparation of Solution 1: ionic crosslinker solution (35.8 grams of alum solution or 40 grams of 25% aq. sodium sulfate solution or 37 grams of 27% aq. sodium silicate solution or 9.26 grams of a 25% aq. solution of trisodium phosphate) in first disposable syringe and Solution 2: polyvinylamine coating solution (20 grams LUPAMIN® 9095, 10 grams PG) in second disposable syringe. Solution 1 was added first, then Solution 2, to the preheated polymer particles. The additions were dropwise over 5 minutes at a Lödige mixing speed of 449 rpm. After complete addition of the coating solutions, the Lödige mixing speed was reduced to 79 rpm, and mixing was continued for 30 minutes.

| | | Propylene | Ionic | | |
|---|---|---|---|---|---|
| | LUPAMIN ® | glycol (PG) | crosslinker | Coating | Residence |
| | 9095 wt % | (wt %) | (wt %) | Temp (° C.) | Time (min.) |
| Control | Base polymer (HySorb B-8700AD) | | | | |
| 4a | 2.0 | 1.0 | 1% Al₂(SO₄)₃ | 60 | 30 |
| 4b | 2.0 | 1.0 | 1% Na | 60 | 30 |
| | | | Silicate | | |
| 4c | 2.0 | 1.0 | 1% Na₂SO₄ | 60 | 30 |
| 4d | 2.0 | 1.0 | 0.1% Na₃PO₄ | 60 | 30 |
| 4e | 2.0 | 1.0 | 0.2% Na₃PO₄ | 60 | 30 |

| | | | | | | Gel | |
|---|---|---|---|---|---|---|---|
| | PSD | | AUL | | | integrity | |
| | (>860μ | CRC | 0.9 psi | FS GBP | GBP 0.3 psi | (1~4 | |
| | wt %) | (g/g) | (g/g) | (Darcies) | (Darcies) | scale) | SAP surface |
| Control | | 26.28 | 19.60 | 85.53 | 8.58 | 1.0 | Hydrophilic |
| 4a | 0.14 | 24.80 | 17.75 | 247.73 | 9.20 | 2.5 | Hydrophilic |
| 4b | 6.86 | 25.58 | 18.95 | 308.50 | 8.64 | 4.0 | Hydrophilic |
| 4c | 0.07 | 25.08 | 18.25 | 291.20 | 7.97 | 4.0 | Hydrophilic |
| 4d | 0.10 | 24.91 | 18.35 | 302.45 | 8.36 | 4.0 | Hydrophilic |
| 4e | 0.67 | 25.21 | 17.62 | 298.66 | 6.72 | 4.0 | Hydrophilic |

Wicking index (cm)

| | 1 min. | 5 min. | 10 min. |
|---|---|---|---|
| Control | 7.0 | 11.0 | 14.5 |
| 4a | 7.0 | 10.5 | 13.0 |
| 4b | 1.5 | 3.0 | 4.2 |
| 4c | 1.0 | 2.5 | 4.3 |
| 4d | 5.5 | 9.0 | 10.5 |
| 4e | 6.2 | 9.5 | 11.0 |

### Example 5

Surface-crosslinked polymer particles, HySorb B-8700AD, were preheated in a laboratory oven at 60° C. When the polymer particles (1 kg) reached 60° C., the particles were transferred to a preheated (60° C.) laboratory Lödige mixer. The polymer particles were maintained at 60° C. throughout the coating step. Preparation of Solution 1: ionic crosslinker solution (varied grams of alum solution) in a first disposable syringe and Solution 2: polyvinylamine coating solution (40 or 20 grams LUPAMIN® 9095, 10 grams PG) in a second disposable syringe. Solution 1 was added first, followed by Solution 2, to the preheated polymer particles. The additions were dropwise over 5 minutes at a Lödige mixing speed of 449 rpm. After complete addition of the coating solution, the Lödige mixing speed was reduced to 79 rpm, and mixing was continued for 30 minutes.

| | | Propylene | | | |
|---|---|---|---|---|---|
| | LUPAMIN ® | glycol (PG) | Aluminum | Coating | Residence |
| | 9095 (wt %) | (wt %) | sulfate (wt %) | Temp (° C.) | Time (min.) |
| Control | Base polymer (HySorb B-8700AD) | | | | |
| 5a | 2.0 | 1.0 | 1.0 | 60 | 30 |
| 5b | 2.0 | 1.0 | 0.7 | 60 | 30 |
| 5c | 2.0 | 1.0 | 0.0 | 60 | 30 |
| 5d | 2.0 | 1.0 | 0.3 | 60 | 30 |

| | | | | | | Gel | |
|---|---|---|---|---|---|---|---|
| | PSD | | AUL | | | integrity | |
| | (>860μ | CRC | 0.9 psi | FS GBP | GBP 0.3 psi | (1~4 | |
| | wt %) | (g/g) | (g/g) | (Darcies) | (Darcies) | scale) | SAP surface |
| Control | | 26.28 | 19.60 | 85.53 | 8.58 | 1.0 | Hydrophilic |
| 5a | 0.14 | 24.80 | 17.75 | 247.7 | 9.20 | 2.5 | Hydrophilic |
| 5b | 0.14 | 24.98 | 18.27 | 251.2 | 9.70 | 4.0 | Hydrophilic |
| 5c | 0.13 | 25.05 | 18.16 | 285.2 | 8.96 | 4.0 | Hydrophilic |
| 5d | 0.14 | 25.64 | 18.67 | 291.1 | 8.79 | 4.0 | Hydrophilic |

Wicking index (cm)

| | 1 min. | 5 min. | 10 min. |
|---|---|---|---|
| Control | 7.0 | 11.0 | 14.5 |
| 5a | 8.0 | 12.0 | 15.0 |
| 5b | 4.0 | 9.0 | 10.0 |
| 5c | 5.5 | 7.0 | 8.5 |
| 5d | 5.5 | 8.0 | 10.0 |

### Example 6

The procedure of Example 5 was repeated to show the absorbency, gel permeability, and gel integrity of HySorb B-8700AD particles coated with LUPAMIN® 9095, propylene glycol, and aluminum sulfate solution.

| | | Propylene | | | |
|---|---|---|---|---|---|
| | LUPAMIN ® | glycol (PG) | Aluminum | Coating | Residence |
| | 9095 (wt %) | (wt %) | sulfate (wt %) | Temp (° C.) | Time (min.) |
| Control | Base polymer (HySorb B-8700AD) | | | | |
| 6a | 1.0 | 1.0 | 0.2 | 60 | 30 |
| 6b | 3.0 | 1.0 | 0.2 | 60 | 30 |
| 6c | 1.0 | 1.0 | 0.8 | 60 | 30 |
| 6d | 3.0 | 1.0 | 0.8 | 60 | 30 |
| 6e | 1.0 | 1.0 | 0.2 | 80 | 30 |
| 6f | 3.0 | 1.0 | 0.2 | 80 | 30 |
| 6g | 1.0 | 1.0 | 0.8 | 80 | 30 |
| 6h | 3.0 | 1.0 | 0.8 | 80 | 30 |
| 6i | 2.0 | 1.0 | 0.5 | 70 | 30 |
| 6j | 2.0 | 1.0 | 0.5 | 70 | 30 |

| | | | | | | Gel | |
|---|---|---|---|---|---|---|---|
| | PSD | | AUL | | | integrity | |
| | (>860μ | CRC | 0.9 psi | FS GBP | GBP 0.3 psi | (1~4 | |
| | wt %) | (g/g) | (g/g) | (Darcies) | (Darcies) | scale) | SAP surface |
| Control | | 26.28 | 19.60 | 85.53 | 8.58 | 1.0 | Hydrophilic |
| 6a | 0.08 | 24.92 | 18.88 | 214.2 | 14.05 | 3.0 | Hydrophobic |
| 6b | 0.07 | 25.81 | 18.29 | 298.0 | 4.61 | 4.0 | Hydrophilic |
| 6c | 0.07 | 25.39 | 20.10 | 206.9 | 10.93 | 1.0 | Hydrophilic |
| 6d | 0.02 | 24.79 | 19.77 | 273.4 | 8.12 | 4.0 | Hydrophobic |
| 6e | 0.31 | 26.23 | 21.26 | 225.5 | 10.65 | 3.0 | Hydrophilic |
| 6f | 0.22 | 25.54 | 20.02 | 298.5 | 5.92 | 4.0 | Hydrophilic |
| 6g | 0.36 | 25.40 | 20.07 | 213.7 | 10.08 | 1.5 | Hydrophilic |
| 6h | 0.28 | 25.09 | 19.04 | 293.1 | 6.30 | 4.0 | Hydrophilic |
| 6i | 0.14 | 25.50 | 20.08 | 251.2 | 9.18 | 4.0 | Hydrophilic |
| 6j | 0.14 | 25.44 | 19.86 | 282.0 | 9.63 | 4.0 | Hydrophilic |

Wicking index (cm)

| | 1 min. | 5 min. | 10 min. |
|---|---|---|---|
| Control | 7.0 | 11.0 | 14.5 |
| 6a | 5.5 | 9.0 | 10.0 |
| 6b | 2.0 | 4.0 | 5.6 |
| 6c | 8.0 | 12.0 | 14.5 |
| 6d | 5.0 | 8.0 | 9.0 |
| 6e | 5.6 | 7.0 | 8.0 |
| 6f | 1.8 | 4.0 | 6.0 |
| 6g | 9.2 | 14.0 | 15.0 |
| 6h | 5.9 | 7.5 | 8.5 |
| 6i | 6.5 | 8.2 | 10.5 |
| 6j | 6.5 | 9.2 | 10.5 |

### Example 7

Surface-crosslinked polymer particles, HySorb B-8700AD, were preheated in a laboratory oven at 80° C. When the polymer particles (1 kg) reached 80° C., the particles were transferred to a preheated (80° C.) laboratory Lödige mixer. The polymer particles were maintained at 80° C. throughout the coating step. Preparation of Solution 1: covalent cross-linker solution (1 or 2 or 3 grams of ethylene glycol diglycidyl ether (EGDGE) in 15 grams of DI water) in a first disposable syringe and solution 2: polyvinylamine coating solution (40 grams LUPAMIN® 9095, 10 grams PG) in a second disposable syringe. Solution 1 was added first, then Solution 2, to the preheated polymer particles dropwise. The additions were over 5 minutes at a Lödige mixing speed of 449 rpm. After complete addition of the coating solution, the Lödige mixing speed was reduced to 79 rpm, and mixing was maintained for 30 minutes.

| | | Propylene | | | |
|---|---|---|---|---|---|
| | LUPAMIN ® | glycol (PG) | | | Coating |
| | 9095 (wt %) | (wt %) | EGDGE (wt %) | H₂O (wt %) | Temp (° C.) |
| Control | Base polymer (HySorb B-8700AD) | | | | |
| 7a | 4.0 | 1.0 | 0.1 | 1.5 | 80 |
| 7b | 4.0 | 1.0 | 0.2 | 1.5 | 80 |
| 7c | 4.0 | 1.0 | 0.3 | 1.5 | 80 |

| | | | | | | Gel | |
|---|---|---|---|---|---|---|---|
| | PSD | | AUL | | | integrity | |
| | (>860μ | CRC | 0.9 psi | FS GBP | GBP 0.3 psi | (1~4 | |
| | wt %) | (g/g) | (g/g) | (Darcies) | (Darcies) | scale) | SAP surface |
| Control | | 26.28 | 19.60 | 85.5 | 8.48 | 1.0 | Hydrophilic |
| 7a | 0.31 | 25.42 | 18.05 | 254.05 | 10.83 | 3.5 | Hydrophilic |
| 7b | 0.54 | 24.72 | 18.05 | 231.31 | 9.69 | 3.0 | Hydrophilic |
| 7c | 0.31 | 24.60 | 18.60 | 188.08 | 9.52 | 2.5 | Hydrophilic |

Wicking index (cm)

| | 1 min. | 5 min. | 10 min. |
|---|---|---|---|
| Control | 7.0 | 11.0 | 14.5 |
| 7a | 4.0 | 5.5 | 6.5 |
| 7b | 5.0 | 7.0 | 8.0 |
| 7c | 6.5 | 9.0 | 10.0 |

### Example 8

Surface-crosslinked polymer particles, HySorb B-8700AD, were preheated in a laboratory oven at 60° C. When the polymer particles (1 kg) reached 60° C., the particles were transferred to a preheated (60° C.) laboratory Lödige mixer. The particles were maintained at a constant 60° C. throughout the coating step. Addition of polyvinylamine coating solution (40 grams LUPAMIN® 9095, 10 grams cosolvent, and 15 grams of DI water) to the preheated polymer particles was performed using a disposable syringe. The addition was dropwise over 5 minutes at a Lödige mixing speed of 449 rpm. After complete addition of the coating solution, the Lödige mixing speed was reduced to 79 rpm, and mixing was continued for 30 minutes. The cosolvents used in this example were: propylene glycol (PG), 1,3-propanediol (PDO), isopropyl alcohol (IPA), methanol (MeOH), and ethylene glycol (EG).

| | LUPAMIN ® | Cosolvent | | Coating | Residence |
|---|---|---|---|---|---|
| | 9095 (wt %) | (wt %) | H₂O (wt %) | Temp (° C.) | Time (min.) |
| 8a | 4.0 | 1% PG | 1.5 | 60 | 30 |
| 8b | 4.0 | 1% PDO | 1.5 | 60 | 30 |
| 8c | 4.0 | 1% IPA | 1.5 | 60 | 30 |
| 8d | 4.0 | 1% MeOH | 1.5 | 60 | 30 |
| 8e | 4.0 | 1% EG | 1.5 | 60 | 30 |

| | | | | | | Gel | |
|---|---|---|---|---|---|---|---|
| | PSD | | | | | integrity | |
| | (>860μ | CRC | AUL 0.9 psi | FS GBP | GBP 0.3 psi | (1~4 | |
| | wt %) | (g/g) | (g/g) | (Darcies) | (Darcies) | scale) | SAP surface |
| 8a | 0.22 | 24.59 | 16.90 | 342.5 | 2.45 | 4.0 | Hydrophobic |
| 8b | 0.23 | 24.65 | 16.39 | 286.5 | 3.76 | 4.0 | Hydrophobic |
| 8c | 0.38 | 23.30 | 16.62 | 289.8 | 3.53 | 4.0 | Hydrophobic |
| 8d | 0.33 | 24.55 | 17.50 | 302.6 | 3.83 | 4.0 | Hydrophobic |
| 8e | 0.70 | 24.64 | 16.60 | 328.9 | 3.00 | 4.0 | Hydrophobic |

Wicking index (cm)

| | 1 min. | 5 min. | 10 min. |
|---|---|---|---|
| 8a | 0.4 | 1.0 | 1.5 |
| 8b | 0.8 | 1.0 | 2.0 |
| 8c | 0.5 | 1.5 | 2.5 |
| 8d | 1.8 | 2.5 | 3.0 |
| 8e | 0.5 | 1.4 | 2.0 |

Examples 1 through 8 show that polyamine-coated SAP particles of the present invention demonstrate excellent permeability (0.3 psi GBP), absorbency is maintained (CRC), and gel integrity (GI) is improved, in addition to a reduced agglomeration of particles when the SAP particle surface is rendered hydrophobic by incorporating a cosolvent in the coating process.

Furthermore, an additional unexpected result is observed with respect to wicking index. Typically, as the wicking index of an SAP particle decreases, the permeability of the SAP particle also decreases. This is attributed to an increase in gel blocking associated with a low wicking index. The present polyamine-coated SAP particles do not exhibit a decrease in permeability properties, even though the wicking index of the polyamine-coated particles may be lower than the wicking index of a control polymer. To the contrary, a decrease in wicking index typically resulted in an increase in permeability properties. Accordingly, the improved absorbance, permeability, and gel integrity properties of the present polyamine-coated SAP particles are independent of the wicking index demonstrated by the particles.

The balanced properties of absorbance, permeability, and gel integrity demonstrated by the present polyamine-coated SAP particles, and the essential independence of these properties from the wicking index, are attributed to the relatively low temperature at which the surface-crosslinked SAP particles are maintained after application of the polyamine to the surfaces of the surface-crosslinked SAP particles. In particular, the low curing temperatures maintain an excellent gel integrity, which is adversely affected by a high temperature cure of the polyamine coating.

The polyamine-coated SAP particles of the present invention are useful as absorbents for water and other aqueous fluids, and particularly can be used as an absorbent component in hygiene articles, such as diapers, tampons, and sanitary napkins. The present polyamine-coated SAP particles also can be used in the following applications, for example: storage, packaging, transportation as a packaging material for water-sensitive articles, for example, flower transportation, and shock protection; food sector for transportation of fish and fresh meat, and the absorption of water and blood in fresh fish and meat packs; water treatment, waste treatment and water removal; cleaning; and agricultural industry in irrigation, retention of meltwater and dew precipitates, and as a composting additive.

Additional applications for the present polyamine-coated SAP particles include medical uses (wound plaster, water-absorbent material for burn dressings or for other weeping wounds, rapid dressings for injuries, rapid uptake of body fluid exudates for later analytical and diagnostic purposes), cosmetics, carrier material for pharmaceuticals and medicaments, rheumatic plaster, ultrasound gel, cooling gel, thickeners for oil/water or water/oil emulsions, textile (gloves, sportswear, moisture regulation in textiles, shoe inserts, synthetic fabrics), hydrophilicization of hydrophobic surfaces, chemical process industry applications (catalyst for organic reactions, immobilization of large functional molecules (enzymes), heat storage media, filtration aids, hydrophilic component in polymer laminates, dispersants, liquefiers), and building construction (sealing materials, systems or films that self-seal in the presence of moisture, and fine-pore formers in sintered building materials or ceramics).

The present invention especially also provides for use of the polyamine-coated SAP particles in an absorption core of hygienic articles. Hygiene articles include, but are not limited to, incontinence pads and incontinence briefs for adults, diapers for infants, catamenial devices, bandages, and similar articles useful for absorbing body fluids.

Hygiene articles, like diapers, comprise (a) a liquid pervious topsheet; (b) a liquid impervious backsheet; (c) a core positioned between (a) and (b) and comprising about 50% to 100% by weight of the present polyamine-coated SAP particles, and 0% to about 50% by weight of hydrophilic fiber material, e.g., a cellulose fiber; (d) optionally a tissue layer positioned directly above and below said core (c); and (e) optionally an acquisition layer positioned between (a) and (c).

Obviously, many modifications and variations of the invention as hereinbefore set forth can be made without departing from the spirit and scope thereof and, therefore, only such limitations should be imposed as are indicated by the appended claims.

1. Superabsorbent polymer particles having a centrifuge retention capacity of at least about 25 g/g, a free swell gel bed permeability of at least 200 Darcies, and a gel integrity of at least 2.

2. The superabsorbent polymer particles of claim 1 further having a gel bed permeability (0.3 psi) of at least 3 Darcies.

3. The superabsorbent polymer particles of claim 1 having a free swell gel bed permeability of at least 250 Darcies.

4. The superabsorbent polymer particles of claim 1 having a gel integrity of at least 2.5.

5. The superabsorbent polymer particles of claim 1 wherein the surfaces of the particle are hydrophobic.

6. The superabsorbent polymer particles of claim 1 wherein the surfaces of the particle are hydrophilic.

7. The superabsorbent polymer particles of claim 1 prepared by a method wherein particles of a surface-crosslinked superabsorbent polymer are coated with a coating composition comprising a polyamine, an optional cosolvent, an optional crosslinking agent, and water;
and maintaining the polyamine-coated polymer particles at 25° C. to 100° C. for about 5 to about 60 minutes.

8. The superabsorbent polymer particles of claim 7 wherein the coating composition comprises a cosolvent, and the superabsorbent polymer particles have a hydrophobic surface.

9. The superabsorbent polymer particles of claim 7 wherein the coating composition is free of an optional cosolvent, and the superabsorbent polymer particles have a hydrophilic surface.

10. The superabsorbent polymer particles of claim 7 wherein the polymer comprises acrylic acid, methacrylic acid, or a mixture thereof.

11. The superabsorbent polymer particles of claim 7 wherein the polymer has a degree of neutralization of about 25 to about 100.

12. The superabsorbent polymer particles of claim 7 wherein the polyamine is present on surfaces of the surface-crosslinked superabsorbent polymer particles in an amount of about 0.1% to about 2%, by weight, of the particle.

13. The superabsorbent polymer particles of claim 7 wherein the polyamine has one or more of primary amino groups, secondary amino groups, tertiary amino groups, and quaternary ammonium groups.

14. The superabsorbent polymer particles of claim 7 wherein the polyamine has a weight average molecular weight of about 5,000 to about 1,000,000.

15. The superabsorbent polymer particles of claim 7 wherein the polyamine is a homopolymer or a copolymer selected from the group consisting of a polyvinylamine, a polyethyleneimine, a polyallylamine, a polyalkyleneamine, a polyazetidine, a polyvinylguanidine, a poly(DADMAC), a cationic polyacrylamide, a polyamine functionalized polyacrylate, and mixtures thereof.

16. The superabsorbent polymer particles of claim 7 wherein the crosslinking agent comprises a salt having (a) a polyvalent metal cation of valence +2, +3, or +4, (b) a polyvalent anion of valence −2, −3, or −4, or (c) a polyvalent cation and a polyvalent anion.

17. The superabsorbent polymer particles of claim 16 wherein the polyvalent metal cation is selected from the group consisting of Mg²⁺, Ca²⁺, Al³⁺, Sc³⁺, Ti⁴⁺, Mn²⁺, Fe^{2+/3+}, CO²⁺, Ni²⁺, Cu^{+/2+}, Zn²⁺, Y³⁺, Zr⁴⁺, La³⁺, Ce⁴⁺, Hf⁴⁺, Au³⁺, and mixtures thereof.

18. The superabsorbent polymer particles of claim 16 wherein the polyvalent anion is selected from the group consisting of sulfate, phosphate, hydrogen phosphate, borate, an anion of a polycarboxylic acid, and mixtures thereof.

19. The superabsorbent polymer particles of claim 7 wherein the crosslinking agent comprises a multifunctional organic component capable of reacting with amino groups of the polyamine.

20. The superabsorbent polymer particles of claim 19 wherein the crosslinking agent is selected from the group consisting of an alkylene carbonate, a polyaziridine, a haloepoxy, a polyisocyanate, a di- or polyglycidyl compound, a alkoxysilyl compound, urea, thiourea, guanidine, dicyandiamide, 2-oxazolidinone or a derivative thereof, bisoxazoline, a polyoxazoline, di- and polyisocyanate, di- and poly-N-methylol compounds, or a compound having two or more blocked isocyanate groups, a multifunctional aldehyde, a multifunctional ketone, a multifunctional acetal, a multifunction ketal, and mixtures thereof.

21. The superabsorbent polymer particles of claim 7 wherein the cosolvent comprises an alcohol, a diol, a triol, or a mixture thereof.

22. The superabsorbent polymer particles of claim 21 wherein the cosolvent comprises methanol, ethanol, propyl alcohol, isopropyl alcohol, ethylene glycol, propylene glycol, an oligomer of ethylene glycol, an oligomer of propylene glycol, glycerin, a monoalkyl ether of propylene glycol, and mixtures thereof.

23. The superabsorbent polymer particles of claim 7 wherein the surface-crosslinked superabsorbent polymer particles comprise a surface-crosslinked polyacrylic acid.

24. The superabsorbent polymer particles of claim 23 wherein the polyamine comprises a polyvinylamine homopolymer or copolymer.

25. A method preparing superabsorbent polymer particles comprising:
(a) providing surface-crosslinked superabsorbent polymer particles;
(b) applying a composition comprising a polyamine, an optional cosolvent, and an optional crosslinking agent to surfaces of the surface-crosslinked polymer particles;
(c) maintaining the coated surface-crosslinked polymer particles of step (b) at about 25° C. to about 100° C. for a sufficient time to provide a cured polyamine coating on the surface-crosslinked polymer particles.

26. The method of claim 25 wherein maintaining step (c) is performed at about 50° C. to about 100° C. for about 5 minutes to about 60 minutes.

27. The method of claim 25 wherein step (b) and step (c) are performed simultaneously.

28. A hygiene article, comprising:
(a) a liquid pervious topsheet;
(b) a liquid impervious backsheet;
(c) a core positioned between (a) and (b), said core comprising about 50% to 100% by weight of the superabsorbent polymer particles of claim 1 and 0% to about 50% by weight of hydrophilic fiber material;
(d) optionally a tissue layer positioned directed above and below said core (c); and
(e) optionally an acquisition layer positioned between (a) and (c).

29. The hygiene article of claim 28 selected from the group consisting of a diaper, a catamenial device, an incontinence pad, an incontinence brief, a bandage, and a burn or wound dressing.
